(19) United States
(12) Patent Application Publication
(10) Pub. No.: US 2008/0279768 A1
(43) Pub. Date: Nov. 13, 2008

(54) Bifunctional Metal Chelating Conjugates

(76) Inventors: Dennis A. Moore, St. Louis, MO (US); Carol P. Howard, Fenton, MO (US)

Correspondence Address: Mallinckrodt Inc., 675 McDonnell Boulevard, HAZELWOOD, MO, 63042, US

(21) Appl. No.: 12/094,995

(22) PCT Filed: Nov. 29, 2006
(86) PCT No.: PCT/US06/45604

§ 371 Date: May 27, 2008

(30) Foreign Application Priority Data
Nov. 29, 2005 (US) 60/740395
Jan. 3, 2006 (US) 60/755759

(51) Int. Cl. A61K 51/10; A61K 103/20; A61K 103/10; A61K 103/34; A61K 51/08; A61K 49/14; A61K 49/16; A61K 49/10; A61K 51/04; A61K 103/40; A61K 103/00

(52) U.S. Cl. 424/1.53; 424/1.69; 424/1.73; 424/9.34; 424/9.35; 424/9.3; 424/1.65

(57) ABSTRACT

The present invention is directed to metal chelating conjugates for use as metallopharmaceutical diagnostics or therapeutic agents. Specifically, conjugates of the present invention include a carrier, a metal coordinating moiety, and a urea linkage chemically linking the metal coordinating moiety to the carrier. The carrier is generally utilized for targeting the conjugate to a biological tissue or organ.

### BACKGROUND

The present invention is generally directed to metal chelating conjugates for use as metallopharmaceutical diagnostic or therapeutic agents.

Metallopharmaceutical diagnostic and therapeutic agents are finding ever-increasing application in biological and medical research, and in diagnostic and therapeutic procedures. Generally, these agents contain a radioisotope or paramagnetic metal, which upon introduction to a subject, become localized in a specific organ, tissue or skeletal structure of choice. When the purpose of the procedure is diagnostic, images depicting in vivo distribution of the radioisotope or paramagnetic metal can be made by various means. The distribution and corresponding relative intensity of the detected radioisotope or paramagnetic metal not only indicates the space occupied by the targeted tissue, but may also indicate a presence of receptors, antigens, aberrations, pathological conditions, and/or the like. When the purpose of the procedure is therapeutic, the agent typically contains a radioisotope, and the radioactive agent delivers a dose of radiation to the local site.

Depending upon the target organ or tissue of interest and the desired diagnostic or therapeutic procedure, a range of metallopharmaceutical agents may be used. One common form is a conjugate including a radioactive or paramagnetic metal, a carrier agent for targeting the conjugate to a specific organ or tissue site, and a linkage for chemically linking the metal to the carrier. In such conjugates, the metal is typically associated with the conjugate in the form of a coordination complex, more typically as a chelate of a macrocycle. See, e.g., Liu, U.S. Pat. No. 6,916,460.

In U.S. Pat. No. 5,435,990, Cheng et al. disclose functionalized macrocyclic polyaminocarboxylate chelants that coordinate rare earth metal ions for use in therapeutic and/or diagnostic oncology procedures. Cheng et al. link their macrocyclic chelant to a carrier agent, in their case an antibody or antibody fragment, via a thiourea linkage. However, thioureas tend to exchange oxygen for sulfur under the reaction conditions for their formation, thereby obscuring the absolute molecular form of the product conjugate. Thiourea linkages may also create a risk of non-specific binding to tissues other than the intended target, which would undesirably result in the delivery of a dose of radiation to the incorrect site.

In U.S. Pat. No. 6,143,274, Tweedle et al. disclose a method for imaging mammalian tissue utilizing a non-ionic complex of a paramagnetic ion of a lanthanide element and a macrocyclic chelating agent. A non-ionic complex, however, is less stable than an anionic complex (i.e., the anionic complex tends to exhibit stronger electrostatic interaction between the cationic metal and anionic ligand).

### SUMMARY

Among the several aspects of the present invention is the provision of conjugates for use in diagnostic and therapeutic procedures. Advantageously, conjugates of the invention may tend to accumulate in the specific organs, tissues or skeletal structures of diagnostic or therapeutic interest with a reduced risk of non-specific binding to non-target tissues, thereby allowing for the conjugates to be targeted to specific disease states, if desired.

In one aspect, the present invention is directed to a conjugate including a metal coordinating moiety and one or more carriers for targeting the conjugate to a biological tissue or organ. In addition, the conjugate includes a linker that includes a urea linkage and that chemically links the metal coordinating moiety to the carrier(s). In some embodiments, a metal (e.g., a radioactive or paramagnetic metal) may be complexed by the metal coordinating moiety of the conjugate.

Another aspect of the invention is directed to a diagnostic or therapeutic method. In this method, a conjugate of the type disclosed herein is administered to a subject (e.g., patient).

Yet another aspect of the invention is directed to a kit for the preparation of a metallopharmaceutical. The kit includes a conjugate of the type disclosed herein.

Still another aspect of the invention is directed to a kit including a protected metal coordinating moiety having an active urea, a deprotecting acid, a buffer, and a solution of a radioactive metal.

Other aspects of the invention will be in part apparent and in part pointed out hereinafter.

### DETAILED DESCRIPTION OF VARIOUS EMBODIMENTS

The present invention provides conjugates that can rapidly form coordination complexes with metals for use in diagnostic or therapeutic metalloradiopharmaceuticals, or magnetic resonance imaging contrast agents. These conjugates may serve as bifunctional chelators (BFC's) for attaching metal ions to bio-directing carriers, sometimes referred to as biomolecules, that bind in vivo to a tissue type, organ or other biologically expressed composition or receptor. Target specific metallopharmaceuticals of the present invention are useful in the diagnosis of disease by magnetic resonance imaging or scintigraphy, or in the treatment of disease by systemic radiotherapy.

Generally, conjugates of the present invention include one or more bio-directing carriers and a metal coordinating moiety covalently joined, directly or indirectly, by a urea moiety. The urea moiety may be directly bonded to the bio-directing carrier(s), or indirectly bonded to the bio-directing carrier(s) through a series of atoms. Similarly and independently, the urea moiety may also be directly bonded to the metal coordinating moiety, or indirectly bonded to the metal coordinating moiety through a series of atoms. Schematically, a conjugate including a bio-directing carrier, the urea moiety, and the metal coordinating moiety of the present invention corresponds to Formula:

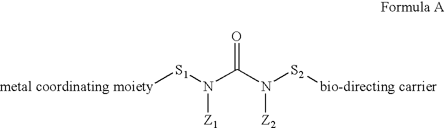

wherein

S₁ and S₂ are spacers, each independently being a bond or a series of atoms, and

Z₁ and Z₂ are independently hydrogen, aryl, C₁₋₇alkyl, C₁₋₇ hydroxyalkyl or C₁₋₇ alkoxyalkyl.

In combination, the sequence —S₁—N(Z₁)C(O)N(Z₂)S₂— may be characterized as a linker, covalently linking the bio-directing carrier to the metal coordinating moiety. Viewed in this manner, the linker includes the urea moiety and spacers, S₁ and S₂, each of the spacers independently being a bond or a series of atoms linking the urea moiety to the metal coordinating moiety or to one or more bio-directing carriers, respectively. Alternatively, however, either or both of the spacers could be considered to be separate and independent components of the conjugate, or members of the metal coordinating moiety and the bio-directing carrier, respectively. For example, S₁ may be considered a part of the metal coordinating moiety and/or S₂ may be considered a part of a bio-directing carrier without departing from the spirit of the present invention.

Although Formula A depicts only a single bio-directing carrier, it is contemplated that a conjugate may include multiple carriers. For instance, in some embodiments, multiple carriers may be connected to the urea linker via S₂. As another example, multiple carriers may be connected to the metal coordinating moiety via a plurality of separate and distinct linkers. In other words, a plurality of linkers may be connected to the metal coordinating moiety, and at least one carrier may be connected with each linker.

Prior to use in a diagnostic and/or therapeutic procedure, a conjugate corresponding to Formula A is generally complexed with a metal to form a metallopharmaceutical diagnostic or therapeutic agent of the present invention.

Bio-Directing Carriers

As previously noted, conjugates of the present invention include one or more bio-directing carriers, also known as biomolecules, that direct the conjugate to the targeted tissue, organ, receptor or other biologically expressed composition. Ideally, each carrier is selective or specific for the targeted organ or tissue site.

Typical bio-directing carriers include hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Specific examples of carriers include steroid hormones for the treatment of breast and prostate lesions; somatostatin, bombesin, CCK, and neurotensin receptor binding molecules for the treatment of neuroendocrine tumors; CCK receptor binding molecules for the treatment of lung cancer; ST receptor and carcinoembryonic antigen (CEA) binding molecules for the treatment of colorectal cancer; dihyroxyindolecarboxylic acid and other melanin producing biosynthetic intermediates for the treatment of melanoma; integrin receptor and atherosclerotic plaque binding molecules for the treatment of vascular diseases; and amyloid plaque binding molecules for the treatment of brain lesions. Exemplary bio-directing carriers also include synthetic polymers such as polyaminoacids, polyols, polyamines, polyacids, oligonucleotides, aborols, dendrimers, and aptamers.

In some embodiments, the bio-directing carrier is selected from among imidazole, triazole, antibodies (e.g., NeutroSpect®, Zevalin®, and Herceptin®, proteins (e.g., TCII, HSA, annexin, and Hb), peptides (e.g., octreotide, bombesin, neurotensin, and angiotensin), nitrogen-containing simple or complex carbohydrates (e.g., glucosamine and glucose), nitrogen-containing vitamins (e.g., vitamin A, B1, B2, B12, C, D2, D3, E, H, and K), nitrogen-containing hormones (e.g., estradiol, progesterone, and testosterone), nitrogen-containing active pharmaceuticals (e.g., celecoxib or other nitrogen-containing NSAIDS, AMD3100, CXCR4 and CCR5 antagonists) and nitrogen-containing steroids. In one example of these embodiments, the bio-directing carrier is selected from among imidazole, triazole, a peptide, a nitrogen-substituted simple or complex carbohydrate, a nitrogen-substituted vitamin, and a nitrogen-substituted small molecule. In another example, the bio-directing carrier may be imidazole, triazole, the N-terminus of a peptide, a nitrogen-substituted simple or complex carbohydrate, or a nitrogen-substituted vitamin. In still another example, the bio-directing carrier (or a terminal group thereof) may be imidazole or triazole.

As mentioned above, some embodiments of the invention may include conjugates having multiple bio-directing carriers. For instance, to increase specificity for a particular target tissue, organ receptor or other biologically expressed composition, multiple bio-directing carriers may be utilized. In such instances, the bio-directing carriers may be the same or different. For example, a single conjugate may possess multiple antibodies or antibody fragments, which are directed against a desired antigen or hapten. Typically, the antibodies used in the conjugate are monoclonal antibodies or antibody fragments that are directed against a desired antigen or hapten. Thus, for example, the conjugate may include two or more monoclonal antibodies having specificity for a desired epitope thereby increasing concentration of the conjugate at the desired site. Similarly, and independently, a conjugate may include two or more different bio-directing carriers each of which is targeted to a different site on the same target tissue or organ. By utilizing multiple bio-directing carriers in this manner, the conjugate advantageously concentrates at several areas of the target tissue or organ, potentially increasing the effectiveness of therapeutic treatment. Further, the conjugate may have a ratio of bio-directing carriers designed to concentrate the conjugate at a target tissue or organ that optimally achieves the desired therapeutic and/or diagnostic results while minimizing non-target deposition.

Linker

As previously noted, one or more bio-directing carriers may be covalently bonded to the metal coordinating moiety via a linker including a urea group. In some embodiments, the linker corresponds to Formula B:

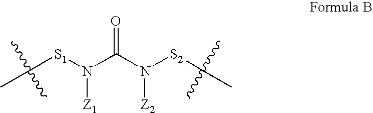

wherein

S₁ and S₂ are independently a covalent bond or a chain of atoms covalently linking the urea moiety to the metal coordinating moiety or to one or more bio-directing carriers, respectively; and

Z₁ and Z₂ are independently selected from the group consisting of hydrogen, aryl, C₁₋₇ alkyl, C₁₋₇ hydroxyalkyl and C₁₋₇ alkoxyalkyl. For example, Z₁ and Z₂ may be selected from the group consisting of hydrogen, C₁₋₇ alkyl, alkoxyalkyl, and phenyl. By way of further example, Z₁ and Z₂ may be selected from a more restrictive group (e.g., hydrogen, C₁₋₄ alkyl and C₁₋₄ alkoxyalkyl). In some embodiments, Z₁ and Z₂ may both be hydrogen.

Whether considered to be part of the linker, separate and independent component(s) of the conjugate, or part of the metal coordinating moiety and bio-directing carrier, respectively, the spacers, S₁ and S₂, are preferably designed to favorably impact biodistribution and potency as well as to provide separation between the metal coordinating moiety and the bio-directing carrier. For example, carbohydrates, polyalkylene glycols, peptides or other polyamino acids, and/or cyclodextrins may be employed as spacers to influence biodistribution of the conjugate, enhance or decrease the rate of blood clearance, and/or direct the route of elimination of the conjugate. In general, preferred spacers are those that result in moderate to fast blood clearance and enhanced renal excretion. Ideally, the spacers are not metabolized via the liver, but instead are cleared by the kidneys thereby diminishing the effects of the conjugates on liver tissue. It should be noted, however, that some embodiments of the invention may include one or more spacers that are metabolized via the liver.

When other than a covalent bond, S₁ and S₂ include a chain of atoms. This chain may be linear, branched, cyclic or a combination thereof. In some embodiments, the chain includes no more than about 40 atoms, or even no more than about 20 atoms. In some embodiments, the chain includes from about 2 to about 15 atoms. The atoms included in this chain are typically selected from the group consisting of carbon, oxygen, nitrogen, sulfur, selenium, silicon and phosphorous. In some embodiments, the atoms may be selected from the group consisting of carbon, oxygen, nitrogen, phosphorous and sulfur. While in other embodiments, the atoms may be selected from the group consisting of carbon, nitrogen, oxygen and phosphorous. In some embodiments, at least some of the chain atoms may be optionally substituted, with exemplary substituents including, but not limited to, one or more hydroxyl, —OR, and R substituents

In some embodiments, for example, S₁ and S₂ are independently a bond (e.g., a single covalent bond), aryl or C₁₋₂₀ alkylene optionally substituted with one or more carbaldehyde, keto, carboxyl (—CO₂H), cyano (—CN), halo, nitro (—NO₂), amido (—C(O)R₁R₂), sulfato (—OSO₃H), sulfito (—SO₃H), phosphato (—OPO₃H₂), phosphito (—PO₃H₂), hydroxyl (—OH), oxy, mercapto (—SH), thio (—SR₁), sulfoxo (S(O)R₁) wherein R, R₁ and R₂ are independently C₁₋₂₀ alkyl optionally substituted with one or more sulfoxo, carboxyl, cyano, nitro, amido, hydroxyl, amino, sulfito, phosphito, sulfato, and phosphate. For these embodiments, each of S₁ and S₂ may independently be a single bond, aryl optionally substituted with one or more of oxy, keto, halo, and amido, or C₁₋₈ alkylene optionally substituted with one or more oxy and keto. In one example of these embodiments, S₁ and S₂ are independently a single bond or C₁₋₄ alkylene optionally substituted with oxy While in another example of these embodiments, S₁ and S₂ are each a single bond.

In some embodiments, S₂ may be: (i) a C₂₋₂₀ alkyl chain or ring optionally substituted with one or more oxygen atoms as ether linkages or pendant with one or more hydroxyl groups as alcohols; (ii) a peptide chain or ring consisting of one or more amino acid residues such as alanine, isoleucine, leucine, valine, phenylalanine, tryptophan, tyrosine, asparagine, methionine, cysteine, serine, glutamine, threonine, aspartic acid, glutamic acid, arginine, histidine, lysine, glycine or proline, conjugated in a natural or unnatural way; or (iii) one or more aromatic rings in chains or condensed in polycycles, optionally substituted with one or more sulfoxo, carboxyl, cyano, nitro, amido, hydroxyl, amino, sulfito, phosphito, sulfato, phosphate, C₁₋₂₀ alkyl chain or ring optionally substituted with one or more sulfoxo, carboxyl, cyano, nitro, amido, hydroxyl, amino, sulfito, phosphito, sulfato, and phosphate.

Metals

Any metal capable of being detected in a diagnostic procedure in vivo or in vitro or useful in the therapeutic treatment of disease can be employed as a metal in conjugates of the present invention. Particularly, any radioactive metal ion or paramagnetic metal ion capable of producing a diagnostic result or therapeutic response in a human or animal body or in an in vitro diagnostic assay may be used. The selection of an appropriate metal based on the intended purpose is known by those skilled in the art. In some embodiments, the metal may be selected from the group consisting of Lu, Lu-177, Y, Y-90, In, In-111, Tc, Tc═O, Tc-99m, Tc-99 m=O, Re, Re-186, Re-188, Re═O, Re-186═O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211. For example, the metal may be selected from the group consisting of Y-90, In-111, Tc-99m, Re-186, Re-188, Cu-64, Ga-67, Ga-68 and Lu-177. By way of another example, the metal may be selected from a more restrictive group (e.g., the group consisting of Y-90, In-111, Tc-99m, Re-186, Cu-64, Ga-67, and Lu-177; or the group consisting of Y-90, In-111, and Tc-99m).

Metal Coordinating Moiety

The metal coordinating moiety may be any moiety used to complex (also referred to as “coordinate”) one or more metals under physiological conditions. Preferably, the metal coordinating moiety forms a thermodynamically and kinetically stable complex with the metal to keep the complex intact under physiological conditions; otherwise, systemic release of the coordinated metal may result.

In general, the metal coordinating moiety may be acyclic or cyclic. For example, metal coordinating moieties include polycarboxylic acids such as EDTA, DTPA, DCTA, DOTA, TETA, or analogs or homologs thereof. To provide greater stability under physiological conditions, however, macrocyclic moieties (e.g., triaza and tetraza macrocycles) are generally preferred. In some embodiments, the macrocyclic metal coordinating moiety is cyclen or tacn.

In some embodiments, the metal coordinating moiety includes a substituted heterocyclic ring where the heteroatom is nitrogen. Typically, the heterocyclic ring includes from about 9 to about 15 atoms, at least 3 of these ring atoms being nitrogen. In one example of these embodiments, the heterocyclic ring includes 3-5 ring nitrogen atoms where at least one of the ring nitrogen atoms is substituted. For these embodiments, the ring carbon atoms may be optionally substituted. One such macrocycle corresponds to Formula (1):

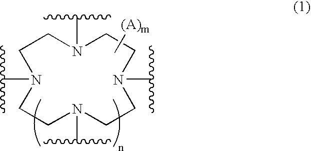

wherein

n is 0, 1 or 2;

m is 0-16, wherein when m is greater than 0, each A is independently selected from the group consisting of optionally substituted C₁₋₂₀alkyl and aryl.

When the metal coordinating moiety corresponds to Formula (1) and m is greater than zero, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio substituents. When A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, sulfato, sulfito, phosphate, phosphito, hydroxyl, oxy, mercapto and thio.

For the metal coordinating moieties of Formula (1), the A substituent, if present, is bonded to any of the ring carbon atoms. Further, each ring carbon atom may be substituted so that the number of possible A substituents varies with the number of ring carbon atoms. In one embodiment of metal coordinating moieties of Formula (1) having at least one A substituent, each A is independently aryl or C₁₋₈ alkyl optionally substituted with one or more aryl, keto, carboxyl, cyano, nitro, C₁₋₂₀ alkyl, amido, sulfato, sulfito, phosphate, phosphito, oxy and thio. For example, each A may be aryl or C₁₋₆ alkyl optionally substituted with one or more aryl, keto, amido and oxy. By way of further example, each A may be methyl.

In general, as the value of n increases, the size of the macrocycle increases. In this manner, the size of the macrocycle may be controlled to match the desired size and coordination number of the metal to be coordinated.

In some embodiments where the metal coordinating moiety includes a substituted heterocyclic ring, the metal coordinating moiety corresponds to Formula (1a)

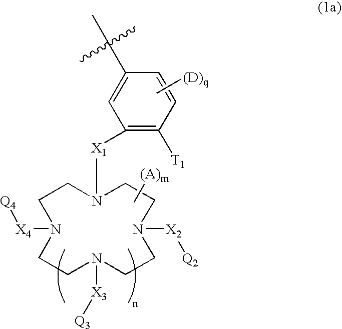

wherein

n is 0, 1 or 2;

m is 0-16, wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;

q is 0-3, wherein when q is greater than 0, each D is independently selected from the group consisting of fluoro, chloro, bromo, iodo, carboxyl, cyano, nitro, amido, hydroxyl, amino, sulfato, sulfito, phosphate, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphate, phosphito, hydroxyl, oxy, mercapto and thio;

Q₂-Q₄ are independently selected from the group consisting of:

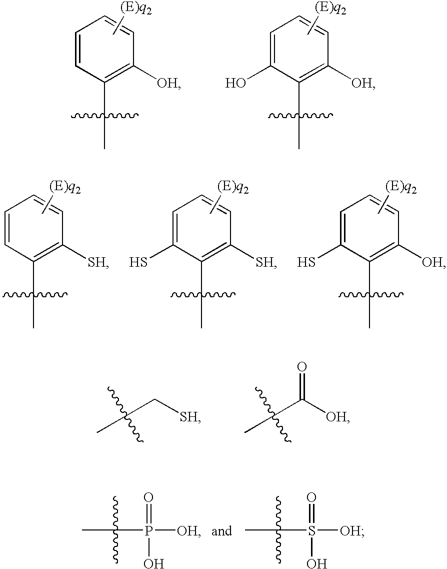

q₂ is 0-4, wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphate; and

T₁ is hydroxyl or mercapto.

For metal coordinating moieties of Formula (1a), the D substituent, if present, is independently bonded to any of the substitutable phenyl ring carbon atoms. In some embodiments, each D may be fluoro, chloro, bromo, iodo, carboxyl, cyano, nitro, amido, hydroxyl, amino, sulfito, phosphito, sulfato, phosphate, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphate. For example, in some embodiments, each D may be bromo, iodo, carboxyl, or hydroxyl. In some embodiments, when T₁ is hydroxyl, D may be a constituent other than hydroxyl at the position that is alpha to the point of attachment of X₁ and beta to the point of attachment of T₁.

For metal coordinating moieties of Formula (1a), the E substituent, if present, is independently bonded to any of the substitutable phenyl ring carbon atoms. In some embodiments, each E may independently be fluoro, chloro, bromo, iodo, carboxyl, cyano, nitro, amido, hydroxyl, amino, sulfito, phosphito, sulfato, phosphato, aryl; or C₁₋₈ alkyl optionally substituted with one or more of C₁₋₂₀ alkyl, carboxyl, cyano, nitro, amido, hydroxyl, amino, sulfito, phosphito, sulfato, and phosphate. For example, in some embodiments, each E may independently be bromo, iodo, carboxyl, or hydroxyl.

Typically, for metal coordinating moieties of Formula (1 a), X₁-X₄ are independently methylene optionally substituted by C₁₋₆ alkyl, halo, or hydroxyl.

In some embodiments of the metal coordinating moieties of Formula (1a), q₂ is 0. Accordingly, Q₂, Q₃, and Q₄ may independently be selected from the group consisting of:

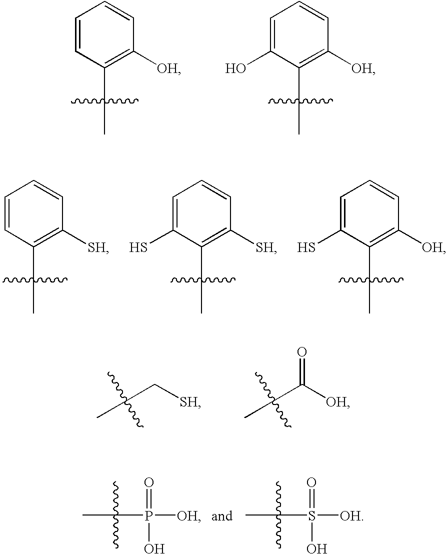

In addition to the metal coordinating moieties including a heterocyclic ring, the metal coordinating moieties may alternatively include a heterosubstituted alkyl chain. Typically, the heterosubstituted alkyl chain includes from about 4 to about 10 atoms in the heterosubstituted alkyl chain, at least 2 of the atoms being nitrogen. In one example of metal coordinating moieties including a heterosubstituted alkyl chain, the chain includes 2-4 nitrogen atoms wherein at least one of the chain nitrogen atoms is substituted. For these embodiments, the chain carbon atoms may optionally be substituted. Typically, the nitrogen atoms including the heterosubstituted alkyl chain are separated from each other by two carbon atoms and thus the metal coordinating moiety may be depicted by the following Formula (2)

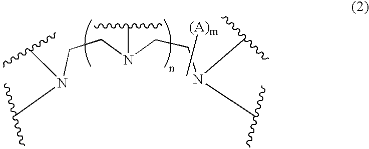

wherein

n is 0, 1 or 2; and

m is 0-8 wherein when m is greater than 0, each A is independently selected from the group consisting of optionally substituted C₁₋₂₀ alkyl and aryl.

When the metal coordinating moiety corresponds to Formula (2) and m is greater than 0, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, sulfato, sulfito, phosphato, phosphito, hydroxyl, oxy, mercapto, or thio substituents. In addition, when A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, sulfato, sulfito, phosphato, phosphito, hydroxyl, oxy, mercapto and thio.

For metal coordinating moieties of Formula (2), the A substituent, if present, may be bonded to any of the ring carbon atoms. Each ring carbon atom may be substituted so that the number of possible A substituents varies with the number of ring carbon atoms. In one embodiment of metal coordinating moieties of Formula (2) having at least one A substituent, each A is independently aryl or C₁₋₈ alkyl optionally substituted with one or more aryl, keto, carboxyl, cyano, nitro, C₁₋₂₀ alkyl, amido, sulfato, sulfito, phosphato, phosphito, oxy and thio. For example, each A may be aryl or C₁₋₆ alkyl optionally substituted with one or more aryl, keto, amido and oxy. By way of further example, each A may be methyl.

In general, as the value of n increases, the length of the heterosubstituted alkyl chain increases. In this manner, the length of the heterosubstituted alkyl chain may be controlled to match the size and coordination capacity of the metal to be coordinated. In some embodiments where the metal coordinating moiety includes a heterosubstituted alkyl chain, the metal coordinating moiety complies with the following Formula (2a)

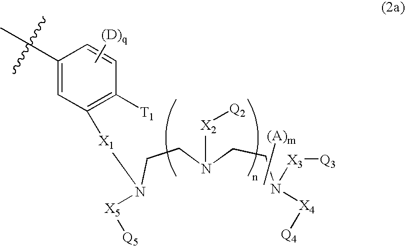

wherein

n is 0, 1 or 2;

m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphate, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphate, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphate, phosphito, hydroxyl, oxy, mercapto and thio;

Q₂-Q₅ are independently selected from the group consisting of:

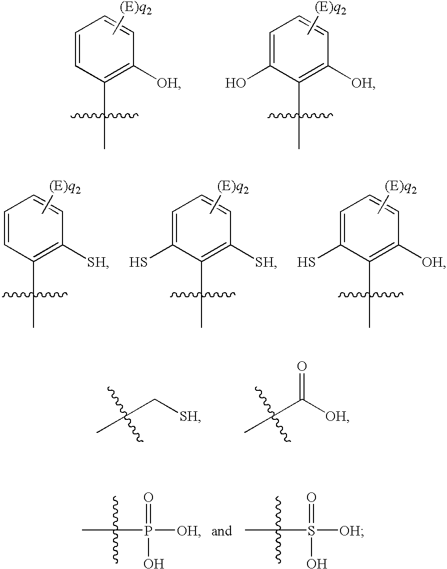

q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphate; and

T₁ is hydroxyl or mercapto.

For metal coordinating moieties of Formula (2a), the D substituent, if present, may be independently bonded to any of the substitutable phenyl ring carbon atoms. In some embodiments, each D may independently be fluoro, chloro, bromo, iodo, carboxyl, cyano, nitro, amido, hydroxyl, amino, sulfito, phosphito, sulfato, phosphate, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphate. For instance, each D of some embodiments may independently be bromo, iodo, carboxyl, or hydroxyl. In some embodiments, when T₁ is hydroxyl, D may be a constituent other than hydroxyl at the position that is alpha to the point of attachment of X₁ and beta to the point of attachment of T₁.

For metal coordinating moieties of Formula (2a), the E substituent, if present, may be independently bonded to any of the substitutable phenyl ring carbon atoms. In some embodiments, each E may independently be fluoro, chloro, bromo, iodo, carboxyl, cyano, nitro, amido, hydroxyl, amino, sulfito, phosphito, sulfato, phosphate, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphate. For instance, each E may independently be bromo, iodo, carboxyl, or hydroxyl in some embodiments.

Typically, for metal coordinating moieties of Formula (2a), X₁-X₄ are independently methylene optionally substituted by C₁₋₆ alkyl, halo, or hydroxyl.

In some embodiments of metal coordinating moieties of Formula (2a), q₂ is 0. Accordingly, Q₂, Q₃, Q₄ and Q₅ are independently selected from the group consisting of:

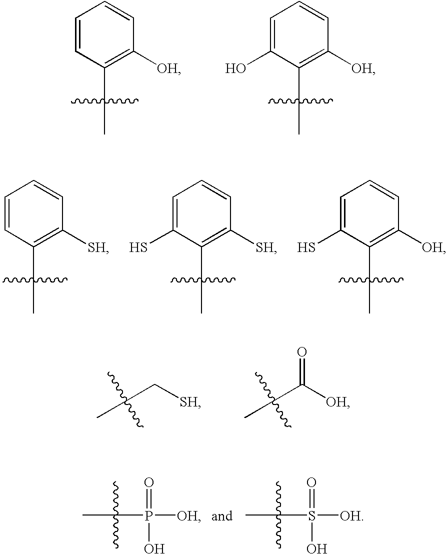

For any of the above embodiments, the metal coordinating moiety may be complexed with a metal, M, thereby forming a metal complex.

In some embodiments where the metal coordinating moiety is a heterocyclic ring and complexed with a metal, M, the complex has the following Formula (3):

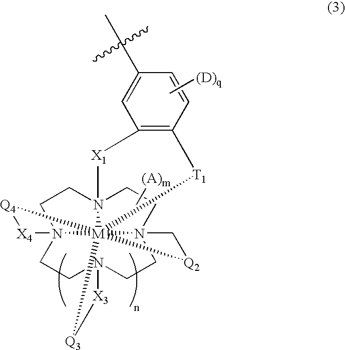

wherein

n is 0, 1 or 2;

m is 0-16 wherein when m is greater than 0, each A is C₁₋₂₀alkyl or aryl optionally substituted by one or more aryl, C₁₋₂₀ alkyl, carbaldehyde, keto, carboxyl, cyano, halo, nitro, amido, sulfato, sulfito, phosphate, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;

Q₂-Q₄ are independently selected from the group consisting of:

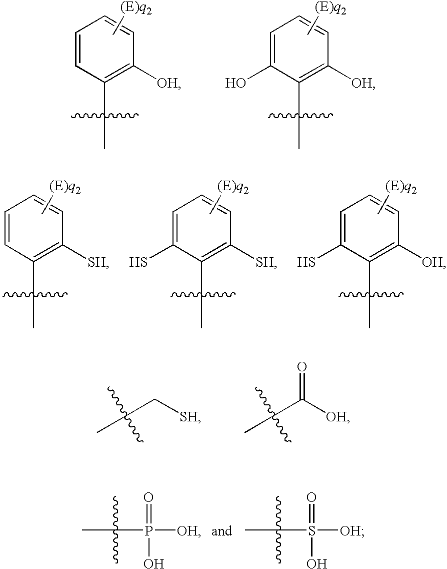

q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato;

T₁ is hydroxyl or mercapto; and

M is selected from the group consisting of Lu, Lu-177, Y, Y-90, In, In-111, Tc, Tc═O, Tc-99m, Tc-99m=O, Re, Re-186, Re-188, Re═O, Re-186=O, Re-188=0, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

In some embodiments where the metal coordinating moiety is a heterosubstituted alkyl chain and is complexed with a metal, M, the complex has the following Formula (4):

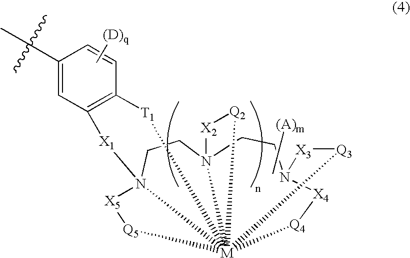

wherein

n is 0, 1 or 2;

m is 0-8 wherein when m is greater than 0, each A is C₁₋₂₀ alkyl or aryl optionally substituted by one or more aryl, C₁₋₂₀alkyl, carbaldehyde, keto, carboxyl, cyano, halo, nitro, amido, sulfato, sulfito, phosphato, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphate, phosphito, aryl, and C₁₋₂₀alkyl optionally substituted with one or more of C₁₋₂₀alkyl, carboxyl, cyano, nitro, amido, hydroxyl, amino, sulfato, sulfito, phosphate, and phosphito;

X₁, X₂, X₃, X₄ and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphate, phosphito, hydroxyl, oxy, mercapto and thio;

Q₂-Q₅ are independently selected from the group consisting of:

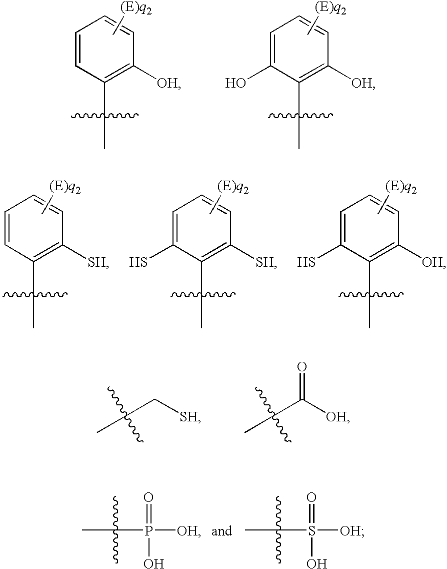

q₂ is 0-4, wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato;

T₁ is hydroxyl or mercapto; and

M is selected from the group consisting of Lu, Lu-177, Y, Y-90, In, In-111, Tc, Tc═O, Tc-99m, Tc-99 m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

Whether the complex corresponds to Formula (3) or Formula (4) typically depends on the particular metal selected for coordination. For example, for yttrium and lanthanides, the complex corresponding to Formula (3) is preferred. Formula (3) is also preferred for iron, copper, and manganese, while Formula (4) is the preferred complex for the remaining transition metals. The preferred complex for any particular metal is related to the potential for transmetallation with endogenous ion. Thus, Formula (3) provides greater stability with high exchange metals, including, but not limited to, yttrium, lanthanides, and gallium. Transmetallation with endogenous ions does not present as great a concern for regular transition metals. While complexes of Formula (3) have been mentioned above as being preferred for use with some metals, while complexes of Formula (4) have been mentioned above as being preferred for use with other metals, it is contemplated that complexes of Formulas (3) and (4) may be utilized with metals other than those listed for the respective complexes.

Macrocyclic metal coordinating moieties with three-dimensional cavities often form metal complexes with high stability. These complexes often exhibit selectivity for certain metal ions based on metal size and coordination chemistry, and capability to adopt a preorganized conformation in the uncomplexed form, which facilitates metal complexation. The selection of appropriate macrocyclic metal coordinating moieties and metals is known by those skilled in the art.

The value of n, and hence the size or length of the metal coordinating moiety, depends upon the particular metal to be coordinated. For yttrium and lanthanides, for example, n is generally 1. For transition metals, n is typically 0 or 1. For manganese and technetium, n is 0, 1, or 2 depending on the value of X₂-X₄. It is, however, contemplated that other values of n may be appropriate for one or more of the metals discussed above.

General Synthesis

For illustrative purposes, the following reaction shows the activation of a metal chelator using carbonyl ditriazine (CDT):

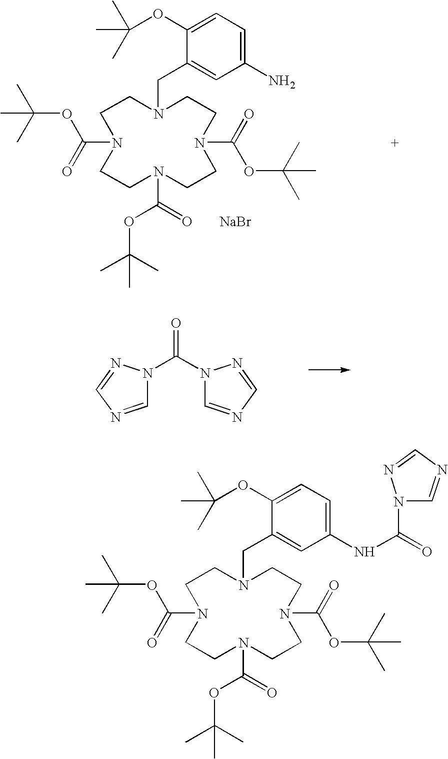

To prevent the reaction of free hydroxyl groups prior to preparation of the conjugate, the hydroxyl groups of the metal coordinating moiety are protected. Any conventional means of protecting the hydroxyl groups is permissible. A variety of protecting groups for the hydroxyl groups and the synthesis thereof may be found in “Protective Groups in Organic Synthesis, 3rd Edition” by T. W. Greene and P. G. M. Wuts, John Wiley and Sons, 1999. Exemplary protecting groups include tert-butyl, methoxymethyl, 1-ethoxymethyl, benzyloxymethyl, (beta-trimethylsilyl ethoxy)methyl, tetrahydropyranyl, 2,2,2-trichloroethyoxycarbonyl, t-butyl(diphenyl)silyl, trialkylsilyl, trichloromethoxycarbonyl and 2,2,2-trichloroethoxymethyl.

To create a reactive urea group from an amine, a mild activating agent is preferred. Exemplary activating agents include carbonyl ditriazine or carbonyl diimidazole (CDI), or mixtures thereof. Other activating agents include phosgene, bis(trichloromethyl)carbonate, and trichloromethyl chloroformate. The reactive intermediates can be isolated as solids, which are stable while under anhydrous conditions. Thus, such an active urea could be allowed to react with a synthetic or natural product (e.g., a biomolecule) to give a protected intermediate. The product may be isolated by precipitation from the reaction mixture using, for example, dichloromethane and ether. Purification of the product can be carried out, for example, by using normal or C18 reverse phase chromatography, as needed. This intermediate can be subsequently deprotected by application of an acid, such as triflic acid in trifluoroethanol, thereby unmasking the phenol hydroxyl and carboxylates.

For this embodiment, the bio-directing carrier and metal may be any of those previously recited. The radioisotope or paramagnetic metal ion is typically dissolved in a solution. The solution may be an aqueous acid or any other solution known in the art to dissolve a radioisotope or paramagnetic metal ion. The solution should allow for the stable storage of the metal in the kit and not interfere with the properties of the metal. Solubilization aids useful in the preparation of radiopharmaceuticals and in the diagnostic kits include, but are not limited to, ethanol, glycerin, polyethylene glycol, propylene glycol, polyoxyethylene sorbitan monooleate, sorbitan monoloeate, polysorbates, poly(oxyethylene)-poly(oxypropylene)-poly(oxyethylene) block copolymers (Pluronics) and lecithin. Preferred solubilizing aids are polyethylene glycol and Pluronics.

Metallopharmaceutical Compositions

Metallopharmaceutical compositions of the present invention include a conjugate, complexed to a metal, dispersed in a pharmaceutically acceptable carrier. The pharmaceutically acceptable carrier, also known in the art as an excipient, vehicle, auxiliary, adjuvant, or diluent, is typically a substance which is pharmaceutically inert, confers a suitable consistency or form to the composition, and does not diminish the therapeutic or diagnostic efficacy of the conjugate. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier tends, at least in part, to be a function of the desired route of administration. In general, metallopharmaceutical compositions of the invention can be formulated for any route of administration so long as the target tissue is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration

Examples of pharmaceutically acceptable carriers for use in compositions of the present invention are well known to those of ordinary skill in the art and may be selected based upon a number of factors: the particular conjugate used, and its concentration, stability and intended bioavailability; the disease, disorder or condition being treated or diagnosed with the composition; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)-lactamide, N,N-dimethylacetamide-amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbateg 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄-C₂₂ fatty acid(s)(e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.)(Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.)(Marcel Dekker, Inc., New York, N.Y., 1980), Remington's Pharmaceutical Sciences (A. Gennaro, ed., 19th ed.)(Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, JOURNAL OF PHARMACEUTICAL SCIENCES, Vol. 52, No. 10, pp. 917-927 (1963).

Dosage

Dosage and regimens for the administration of the pharmaceutical compositions of the invention can be readily determined by those with ordinary skill in diagnosing or treating disease. It is understood that the dosage of the conjugates will be dependent upon the age, sex, health, and weight of the recipient, kind of concurrent treatment, if any, frequency of treatment, and the nature of the effect desired. For any mode of administration, the actual amount of conjugate delivered, as well as the dosing schedule necessary to achieve the advantageous effects described herein, will also depend, in part, on such factors as the bioavailability of the conjugate, the disorder being treated or diagnosed, the desired therapeutic or diagnostic dose, and other factors that will be apparent to those of skill in the art. The dose administered to an animal, particularly a human, in the context of the present invention should be sufficient to affect the desired therapeutic or diagnostic response in the animal over a reasonable period of time.

Radiolabeled scintigraphic imaging agents provided by the present invention are provided having a suitable amount of radioactivity. In forming diagnostic radioactive complexes, it is generally preferred to form radioactive complexes in solutions containing radioactivity at concentrations of from about 0.01 millicurie (mCi) to 100 mCi per mL. Generally, the unit dose to be administered has a radioactivity of about 0.01 mCi to about 100 mCi, preferably about 1 mCi to about 30 mCi. The solution to be injected at unit dosage is from about 0.01 mL to about 10 mL. The amount of radiolabeled conjugate appropriate for administration is dependent upon the distribution profile of the chosen conjugate in the sense that a rapidly cleared conjugate may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at the non-target tissue.

Typically, an In-111 diagnostic dose is 3-6 mCi while a typical Tc-99m does is 10-30 mCi. Generally, radiotherapeutic doses of radiopharmaceuticals vary to a greater extent, depending on the tumor and number of injections of cycles. For example, cumulative doses of Y-90 range from about 100-600 mCi (20-150 mCi/dose), while cumulative doses of Lu-177 range from about 200-800 mCi (50-200 mCi/dose).

Kits

For convenience, metallopharmaceutical compositions of the present invention may be provided to the user in the form of a kit containing some or all of the necessary components. The use of a kit is particularly convenient since some of the components, e.g., a radioisotope, have a limited shelf life, particularly when combined. Thus, the kit may include one or more of the following components (i) a conjugate, (ii) a metal coordinated to or for coordination by the conjugate, (iii) a carrier solution, and (iv) instructions for their combination and use. Depending on the metal, a reducing agent may be necessary to prepare the metal for reaction with the conjugate. Exemplary reducing agents include Ce (III), Fe (II), Cu (I), Ti (III), Sb (III), and Sn (II). Of these, Sn (II) is particularly preferred. Often the components of the kit are in unit dosage form (e.g., each component in a separate vial).

For reasons of stability, it may be preferred that the conjugate be provided in a dry, lyophilized state. The user may then reconstitute the conjugate by adding the carrier or other solution.

Because of the short half-life of suitable radionuclides, it will frequently be most convenient to provide the kit to the user without a radionuclide. The radionuclide is then ordered separately when needed for a procedure. Alternatively, if the radionuclide is included in the kit, the kit will most likely be shipped to the user just before it is needed.

In addition to the metal coordinating moiety, biomolecule, active urea, metal and deprotecting acid, the kit of the present invention typically includes a buffer. Exemplary buffers include citrate, phosphate and borate.

The kit optionally contains other components frequently intended to improve the ease of synthesis of the radiophammaceutical by the practicing end user, the ease of manufacturing the kit, the shelf-life of the kit, or the stability and shelf-life of the radiopharmaceutical. Such components of the present invention include lyophilization aids, e.g., mannitol, lactose, sorbitol, dextran, Ficoll, and polyvinylpyyrolidine (PVP); stabilization aids, e.g., ascorbic acid, cysteine, monothioglycerol, sodium bisulfite, sodium metabisulfite, gentisic acid, and inositol; and bacteriostats, e.g., benzyl alcohol, benzalkonium chloride, chlorbutanol, and methyl, propyl, or butyl paraben.

Typically, when the conjugate is formulated as a kit, the kit includes multiple vials consisting of a protected metal coordinating moiety having an active urea group, a deprotecting acid, a buffer, and a solution of a radioactive metal such as, but not limited to, In-111, Y-90 or Lu-177. In practice, the user will take the vial containing the metal coordinating moiety and add a solution of a bio-directing carrier of interest bearing a reactive amino (NH₂) group. Once conjugation is complete, the deprotecting acid is added to affect deprotection, followed by addition of the radioactive metal. The mixture is then buffered to complete complexation of the radioactive metal by the metal chelator.

### DEFINITIONS

The compounds described herein may have asymmetric centers. Compounds of the present invention containing an asymmetrically substituted atom may be isolated in optically active or racemic form. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The present invention includes all isotopes of atoms occurring in the present compounds. Isotopes include those atoms having the same atomic number but different mass numbers.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “amido” as used herein includes substituted amido moieties where the substituents include, but are not limited to, one or more of aryl and C₁₋₂₀alkyl, each of which may be optionally substituted by one or more aryl, carbaldehyde, keto, carboxyl, cyano, halo, nitro, C₁₋₂₀ alkyl, sulfato, sulfito, phosphato, phosphito, hydroxyl, oxy, mercapto, and thio substituents.

The term “amino” as used herein includes substituted amino moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, sulfato, sulfito, phosphato, phosphito, hydroxyl, oxy, mercapto, and thio substituents.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “complex” refers to a metal coordinating moiety of the invention, e.g. Formula (1), complexed or coordinated with a metal. The metal is typically a radioactive isotope or paramagnetic metal ion.

The term “conjugate” refers to a metal coordinating moiety of the invention, e.g. Formula (1), bonded to a bio-directing carrier (biomolecule) whether or not the metal coordinating moiety is complexed with a metal. For the present invention, the metal coordinating moiety is bonded to the bio-directing carrier directly or indirectly by a urea moiety.

The terms “halogen” or “halo” as used herein alone or as part of another group refer to chlorine, bromine, fluorine, and iodine.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring. The heterocyclo group preferably has 1 to 5 nitrogen atoms in the ring, and may be bonded to the remainder of the molecule through a carbon atom. Exemplary heterocyclics include macrocyclics, cyclen, tacn, DOTA, DOTMA, DOTP, and TETA.

The “heterosubstituted alkyl” moieties described herein are alkyl groups in which a carbon atom is covalently bonded to at least one heteroatom and optionally with hydrogen, the heteroatom being, for example, a nitrogen atom.

The term “metallopharmaceutical” as used herein refers to a pharmaceutically acceptable compound including a metal, wherein the compound is useful for imaging or treatment.

### EXAMPLES

### Example 1

### Synthesis of 1,4,7,10-Tetraazacyclododecane-1,4,7-triacetic acid, 10-[(2-butoxy-5-(N-({CNCbl-5′-[(13-amino)-4,7,10-trioxamidencanecarbamate])}-carbonylamino)phenyl)methyl], tri-butyl ester (8)

### Synthesis of 2-t-butoxy-5-nitrobenzyl bromide (1)

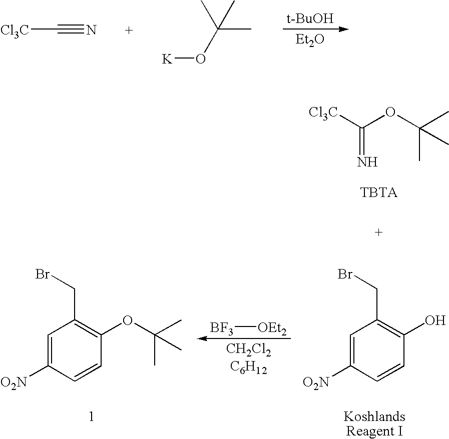

t-Butyl trichloroacetimidate (TBTA)

Potassium t-butoxide (1M in t-butanol), 69 mL 0.069 mole, was dissolved in diethyl ether, 69 mL to form a solution. This solution was added dropwise, over 30 minutes, to a 0° C. solution of trichloroacetonitrile, 100 g 0.69 mole, in diethyl ether, 69 mL. The mixture was allowed to warn to room temperature over one hour and stirred for an additional hour with heating at reflux. The mixture was cooled to room temperature and evaporated under reduced pressure to an oil. The oil was dissolved in hexanes, 140 mL, and filtered to remove potassium salts. The filtrate was then evaporated under reduced pressure and the residue vacuum distilled collecting the fraction distilling at 2.4 mm Hg and 40° C. Yield 105 g, 69% based on trichloroacetonitrile. ¹H nmr (300 MHz CDCl₃): 1.58, (s, 9H), 8.21 (br, s, 1H). ¹³C (75.45 MHz, CDCl₃) 27.23, 83.86, 92.78, 160.33.

### 2-t-Butoxy-5-nitrobenzyl bromide (1)

A suspension of 2-hydroxy-5-nitrobenzylbromide, 19.4 g 0.0836 mole, cyclohexane, 334 mL, and dichloromethane, 167 mL, was stirred under nitrogen. To this suspension was added a solution of t-butyl trichloroacetimidate, 73.08 g 0.334 mole, in cyclohexane, 669 mL, dropwise over 3.5 hours. The mixture was stirred for one hour after completion of the addition and boron trifluoride etherate, 200 μL, was added. The mixture was allowed to stir overnight. A large amount of precipitate, trichloroacetamide, formed. The reaction mixture was treated with sodium bicarbonate, 4.00 g 0.0418 mole, stirred for one hour and filtered. The solids were washed with diethyl ether and the combined filtrates concentrated to an oil under reduced pressure. The oil was treated with hexanes, 100 mL, and the solution stirred until crystals formed. After cooling to −20° C. and stirring for an additional hour, the resulting solid was collected by filtration, washed with cold, fresh hexane, suctioned dry and vacuum dried. Yield 13.2 g, 55% based on 2-hydroxy-5-nitrobenzyl bromide. Calc C, 45.85; H, 4.90; N, 4.86, Br 27.73. Found C, 45.39; H, 5.07; N, 4.94, Br 27.66. ¹H nmr (300 MHz CDCl₃) 1.58 (s, 9H), 4.48 (s, 2H), 7.10 (d, JH=9 Hz, 1H), 8.11 (dd, J=9 Hz, J=2.7 Hz, 1H), 8.22 (d, J=2.7 Hz, 1H). ¹³C (75.45 MHz, CDCl₃) 28.92, 81.59, 116.86, 125.07, 126.34, 129.98, 140.69, 159.97.

### Synthesis of 1,4,7,10-Tetraazacyclododecane-1,4,7-triacetic acid, 10-[(2-t-butoxy-5-nitrophenyl)methyl]-, tri-t-butyl ester (3)

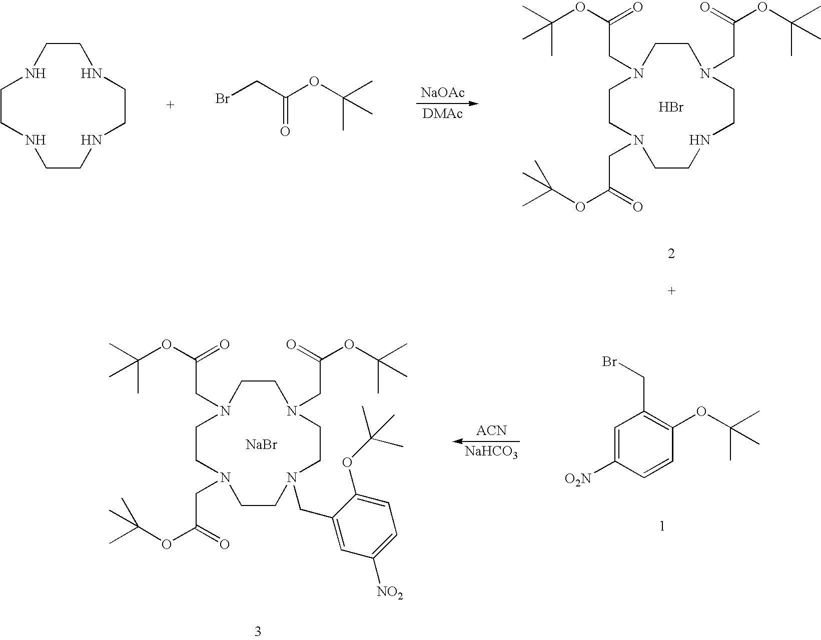

### Synthesis of 1,4,7,10-Tetraazacyclododecane-1,4,7-triacetic acid, tri-t-butyl ester Hydrobromide (2)

Cyclen, 32.0 g 0.186 mole, and sodium acetate trihydrate, 75.8 g 0.557 mole, were stirred with dimethylacetamide, 600 mL, for one hour. To this mixture was added a solution of t-butyl bromoacetate, 109 g 0.557 mole, in dimethylacetamide, 150 mL, dropwise, over four hours. The rate of the addition was adjusted so as to keep the temperature of the reaction mixture less than 25° C. The mixture was allowed to stir over two nights. After cooling to −10° C. and stirring for two hours, the resulting solid was collected by filtration, washed with cold, fresh dimethylacetamide, 50 mL, and suctioned dry. The solid was dissolved in chloroform, 0.5 L, and the solution washed with water, 3×200 mL. The organic phase was collected, dried with magnesium sulfate, filtered and concentrated, under reduced pressure, to 300 mL. Hexanes, 300 mL, was added and the solution stirred for one hour at room temperature. After a few minutes crystallization began. The resulting slurry was cooled to −20° C., stirred for two hours and filtered. The solid was washed with cold, fresh chloroform-hexanes, 50 mL 1:1, suctioned dry and vacuum dried overnight at room temperature. Yield 69 g, 62% based on cyclen. ¹H nmr (300 MHz, CDCl₃) 1.44 (s, 9H), 1.45 (s, 18H), 2.87-2.90 (br, m, 12H), 3.07-3.08 (br, m, 4H), 3.27 (s, 2H), 3.56 (s, 4H), 9.97 (br,s, 2H). ¹³C nmr (75.45 MHz, CDCl₃) 28.15, 28.18, 47.44, 48.68, 49.11, 51.15, 51.25, 58.11, 81.54, 81.70, 169.32, 170.21.

### Synthesis of 1,4,7,10-Tetraazacyclododecane-1,4,7-triacetic acid, 10-[(2-t-butoxy-5-nitrophenyl)methyl]-, tri-t-butyl ester, sodium bromide complex (3)

1,4,7,10-Tetraazacyclododecane-1,4,7-triacetic acid, tri-t-butyl ester hydrobromide, 8.46 g 0.0142 mole, was stirred with aqueous sodium hydroxide, 0.1N 200 mL, and diethyl ether, 200 mL. When the entire solid had dissolved, the organic phase was collected and the aqueous phase washed with diethyl ether, 2×200 mL. The combined organic extracts were dried with magnesium sulfate, filtered and evaporated, under reduced pressure, to an oil. The oil was dissolved in acetonitrile, 135 mL. To this solution was added sodium bicarbonate, 1.19 g 0.0142 mole, followed by 2-t-butoxy-5-nitrobenzyl bromide, 4.50 g 0.0156 mole. The mixture was warmed to 35° C., and stirred overnight under argon. When the reaction was complete by nmr, 12-14 hours total, the mixture was filtered and the filtrate concentrated under reduced pressure to give an oil. The oil was suspended in diethyl ether, 50 mL, and a white precipitate formed after stirring. The solid was collected by filtration, suctioned dry and dried in a vacuum overnight. Yield 11.7 g, 98% based on starting 1,4,7,10-Tetraazacyclododecane-1,4,7-triacetic acid, tri-t-butyl ester hydrobromide. Anal. Calc. C, 52.73; H, 7.77; N, 8.31, Br 9.48. Found C, 52.31; H, 7.68; N, 8.26, Br 9.67. ¹H nmr (300 MHz, CDCl₃) 1.45 (s, 27H), 1.51 (s, 9H), 1.78 (br, s, 2H), 2.20 (m, 4H), 2.33 (br, 4H), 2.73 (br, 4H), 2.93 (complex, br, 6H), 3.10 (m, 2H), 3.29 (s, 1H), 3.37 (s, 1H), 3.57 (s, 2H), 7.15 (d, ³J_{H-H}=9 Hz, 1H), 8.07 (d of d, ³J_{H-H}=9 Hz, ⁴J_{H-H}=2.7 Hz, 1H), 8.88 (d, ⁴J_{H-H}=2.7 Hz). ¹³C nmr (75.45 MHz, CDCl₃) 28.15, 28.20, 29.48, 50.00 (br), 55.97, 56.28, 81.83, 82.68, 83.29, 118.27, 124.18, 127.44, 131.13, 141.95, 161.31, 172.67, 173.62.

### Synthesis of 1,4, 7,10-Tetraazacyclododecane-1,4,7-triacetic acid, 10-[(2-t-butoxy-5-(triazolyl- and imidazolylcarbonylamino)phenyl)methyl]-, tri-t-butyl ester (5) and (6)

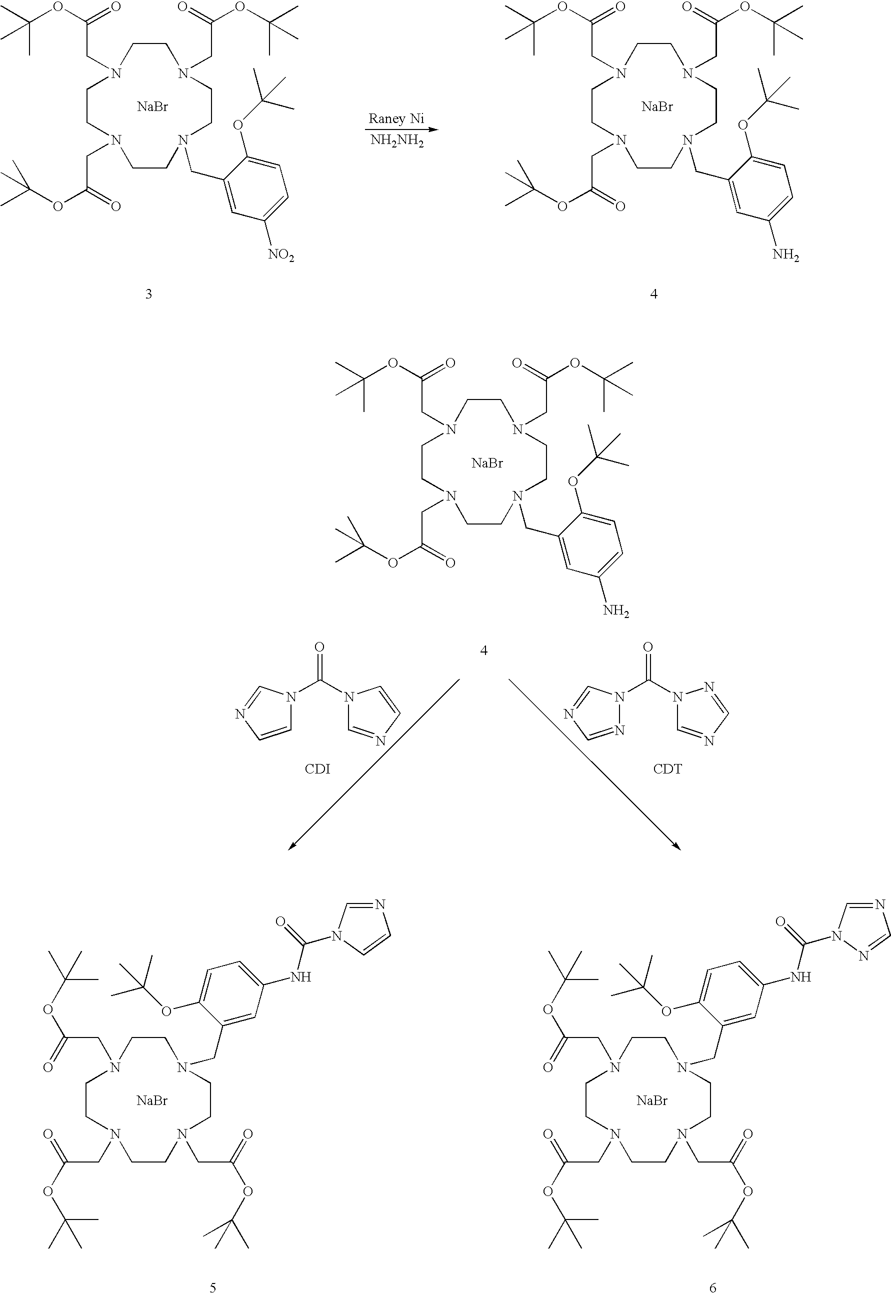

### Synthesis of 1,4,7,10-Tetraazacyclododecane-1,4,7-triacetic acid, 10-[(2-t-butoxy-5-aminophenyl)methyl]-, tri-t-butyl ester, sodium bromide complex, pentahydrate (4)

Raney nickel-water slurry, ca. 0.4 g, methanol, 20 mL, and hydrazine hydrate, 1.15 mL, were placed in an argon-flushed 250 mL round bottom flask. The mixture was heated to reflux and a solution consisting of 1,4,7,10-tetraazacyclododecane-1,4,7-triacetic acid, 10-[(2-t-butoxy-5-nitrophenyl)methyl]-, tri-t-butyl ester, sodium bromide complex, 4.00 g 0.0047 mole, methanol, 20 mL, was added dropwise. The addition took 30 minutes. The mixture was heated for an additional 10 minutes. An aliquot was removed, evaporated and dissolved in CDCl₃. ¹H nmr showed the reaction to be greater than 95 mole % complete. The reaction mixture was cooled to room temperature, filtered on celite. The filtrate was evaporated and dissolved in chloroform, 14 mL, filtered to remove some fine solids and treated with diethyl ether, 80 mL. After stirring for a few minutes, crystallization began. The mixture was cooled to −10° C., stirred for one hour and the solid collected by filtration, washed with fresh ether, suctioned dry and vacuum dried. Yield 3.57 g 85%, based on starting 1,4,7,10-tetraazacyclododecane-1,4,7-triacetic acid, 10-[(2-t-butoxy-5-nitrophenyl)methyl]-, tri-t-butyl ester, sodium bromide complex. Anal. Calc. C, 50.22; H, 8.54; N, 7.91, Br 9.03. Found C 50.49, H 7.68, N 7.80, Br 8.86. ¹H nmr (300 MHz, CDCl₃) 1.28 (s, 9H), 1.45 (s, 9H), 1.47 (s, 18H), 2.22 (m, 4H), 2.36 (br, 6H), 2.80 (br, 6H), 2.97 (s, br, 4H), 3.40 (s, 2H), 3.44 (s, 2H), 6.45 (d of d, ³J_{H-H}=9 HZ, ⁴J_{H-H}=2.7 Hz, 1H), 6.75 (d, ³J_{H-H}=9 Hz, 1H), 6.92 (d, ⁴J_{H-H}=2.7 Hz). ¹³C nmr (75.45 MHz, CDCl₃) 28.25, 28.41, 29.50, 50.00 (br), 54.00, 56.15, 56.49, 79.43, 82.55, 82.88, 115.03, 117.73, 124.07, 131.90, 142.69, 146.74, 172.64, 173.38.

### Synthesis of 1,4,7,10-Tetraazacyclododecane-1,4,7-triacetic acid, 10-[(2-t-butoxy-5-(imidazolylcarbonylamino)phenyl)methyl]-, tri-t-butyl ester, sodium bromide complex (5)

1,4,7,10-Tetraazacyclododecane-1,4,7-triacetic acid, 10-[(2-t-butoxy-5-aminophenyl)methyl]-, tri-1-butyl ester, sodium bromide complex, pentahydrate, 1.00 g 0.0011 mole, was dissolved in dichloromethane, 4 mL, and carbonyldiimidazole, 0.26 g 0.16 mole, was added. ¹H nmr showed the reaction to be complete by disappearance of the aniline chemical shifts. The mixture was evaporated and the resulting oil stirred with diethyl ether, 25 mL. The resulting solid was collected by filtration, washed with fresh ether and vacuum dried. Yield 0.77 g, 77% based on starting 1,4,7,10-tetraazacyclododecane-1,4,7-triacetic acid, 10-[(2-t-butoxy-5-aminophenyl)methyl]-, tri-t-butyl ester, sodium bromide complex, pentahydrate. ¹H nmr (300 MHz, CDCl₃) 1.33 (s, 9H), 1.44 (s, 27H), 2.24 (br, m, 6H), 2.58 (br, m, 10H), 3.00 (br, s, 2H), 3.05 (br, s, 4H), 3.72 (s, 2H), 7.02 (d, ³J_{H-H}=8.7 Hz, 1H), 7.06 (s, 1H), 7.88 (d of d, ³J_{H-H}=8.7 Hz, ⁴J_{H-H}=2.1 Hz, 1H), 8.01 (d, ⁴J_{H-H}=2.1 HZ, 1H), 8.52 (s, 1H), 8.57 (s, 1H), 10.59 (br, s, 1H). ¹³C nmr (75.45 MHz, CDCl₃) 28.24, 28.34, 29.64, 50.9(br), 56.13, 56.70, 79.92, 82.84, 83.00, 117.92, 122.30, 122.42, 126.10, 128.32, 130.02, 132.57, 137.26, 147.82, 151.59, 172.41, 173.11.

### Synthesis of 1,4,7,10-Tetraazacyclododecane-1,4,7-triacetic acid, 10-[(2-t-butoxy-5-(triazolylcarbonylamino)phenyl)methyl]-, tri-t-butyl ester (6)

Carbonyldi-1,2,4-triazole, 0.14 g 0.0009 mole, was dissolved in dichloromethane, 10 mL. To this was added, dropwise, a dichloromethane, 5 mL, solution of 1,4,7,10-tetraazacyclododecane-1,4,7-triacetic acid, 10-[(2-t-butoxy-5-aminophenyl)methyl]-, tri-t-butyl ester, sodium bromide complex, pentahydrate, 0.50 g 0.0006 mole. The mixture was allowed to stir for two hours and diethyl ether, 50 mL, was added to precipitate the product. Yield 0.3 g 60% based on 1,4,7,10-tetraazacyclododecane-1,4,7-triacetic acid, 10-[(2-t-butoxy-5-aminophenyl)methyl]-, tri-t-butyl ester, sodium bromide complex, pentahydrate. ¹H nmr (300 MHz, CDCl₃) 1.38 (s, 9H), 1.44 (s, 27H), 2.19 (br, m, 4H), 2.38 (br, m, 4H), 2.80 (br, m, 8H), 3.00 (s, 6H), 3.57 (s, 2H), 7.12 (d, ³J_{H-H}=8.1 Hz, 1H), 7.72 (d of d, ³J_{H-H}=8.7 HZ, ⁴J_{H-H}=2.7 Hz, 1H), 7.84 (4J_{H-H}=2.7 Hz, 1H), 7.91 (s, 1H), 8.87 (s, 1H). ¹³C nmr (75.45 MHz, CDCl₃) 28.23, 28.44, 29.54, 49.9(br), 56.01, 56.44, 80.39, 82.56, 83.07, 119.75, 122.96, 123.01, 131.03, 132.23, 143.57, 144.71, 152.30, 152.70, 172.66, 173.72.

### 1,4,7,10-Tetraazacyclododecane-1,4,7-triacetic acid, 10-[(2-t-butoxy-5-(N-{CNCbl-5′-[(13-amino)-4,7,10-trioxa-tridecanecarbamate])}-carbonylamino)phenyl)methyl](9)

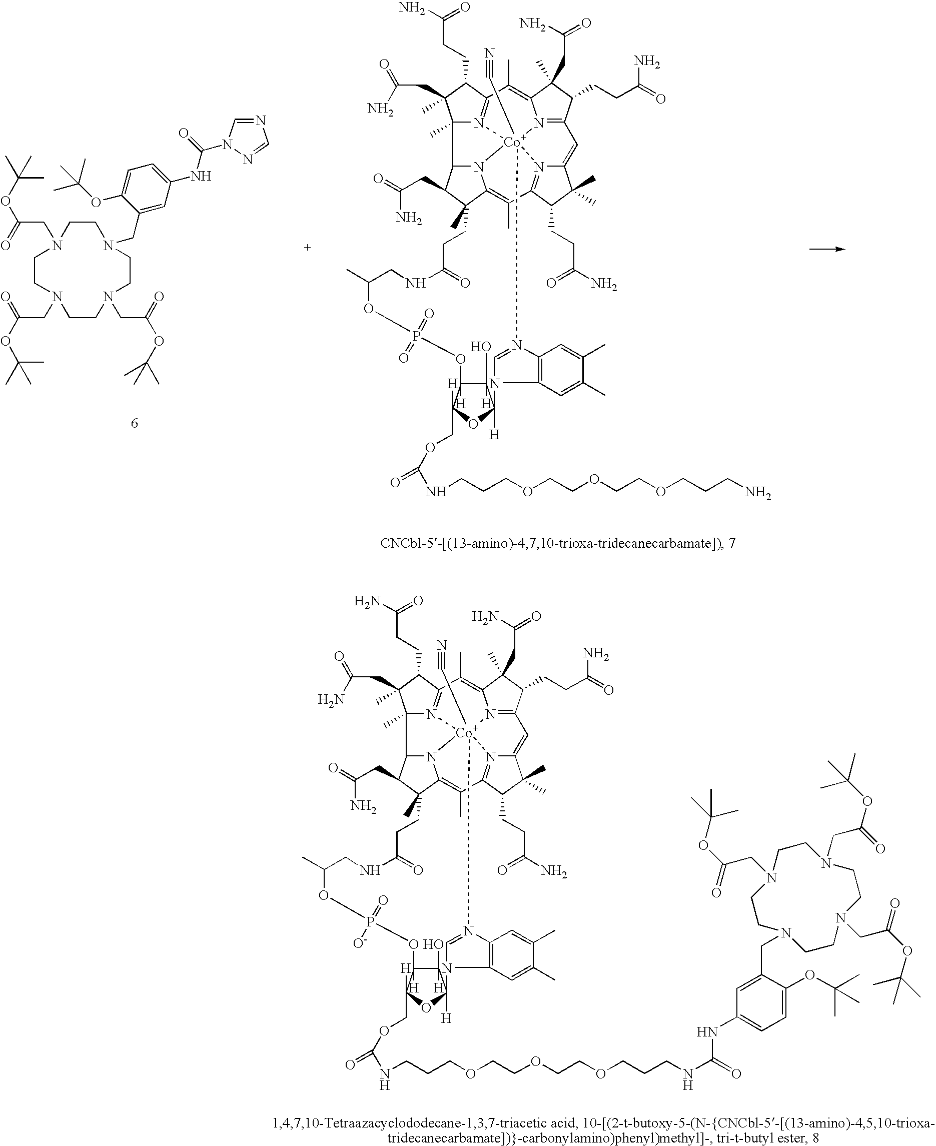

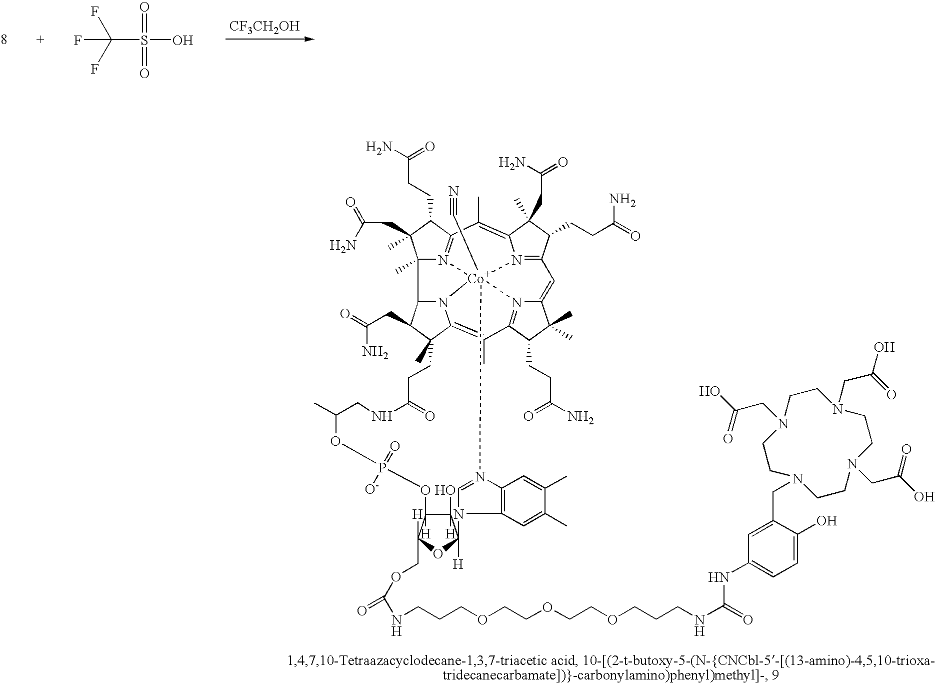

### Synthesis of 1,4,7,10-Tetraazacyclododecane-1,4,7-triacetic acid 10-[(2-t-butoxy-5-(N-{CNCbl-5′-[(13-amino)-4,7,10-trioxa-tridecanecarbamate])}-carbonylamino)phenyl)methyl]-, tri-t-butyl ester (8)

CNCbl-5′-[(13-amino)-4,7,10-trioxa-tridecanecarbamate] was dissolved in dry dimethylsulfoxide, 1.0 mL, under argon. To this was added 1,4,7,10-tetraazacyclododecane-1,4,7-triacetic acid, 10-[(2-t-butoxy-5-(triazolylcarbonylamino)phenyl)methyl]-, tri-t-butyl ester, 0.10 g 0.0001 mole. The mixture was allowed to until HPLC showed the reaction was complete. The mixture was precipitated using dichloromethane, 5 mL, and collected by filtration. The crude solid was washed with fresh dichloromethane, 25 mL, diethyl ether, 25 mL, and suctioned dry. The solid was dissolved in methanol and purified by C-18 column chromatography. Pure fractions were combined, concentrated under reduced pressure and the product isolated by precipitation with acetone. Yield 0.12 g 41% based on starting 1,4,7,10-tetraazacyclododecane-1,4,7-triacetic acid, 10-[(2-t-butoxy-5-(N-{CNCbl-5′-[(13-amino)-4,7,10-trioxa-tridecanecarbamate])}-carbonylamino)phenyl)methyl]-, tri-t-butyl ester. The product was characterized by HPLC, single eluting peak at 15 min, and mass spectrometry (M+3H)³⁺=774.1 theory=774.1.

### Synthesis of 1,4,7,10-Tetraazacyclododecane-1,4,7-triacetic acid, 10-[(2-hydroxy-5-(N-{CNCbl-5′-[(13-amino)-4,7,10-trioxa-tridecanecarbamate])}-carbonylamino)phenyl)methyl]-(9)

1,4,7,10-Tetraazacyclododecane-1,4,7-triacetic acid, 10-[(2-t-butoxy-5-(N-{CNCbl-5′-[(13-amino)-4,7,10-trioxa-tridecanecarbamate])}-carbonylamino)phenyl)methyl]-, tri-t-butyl ester, 0.003 g 1.31 μmoles, was dissolved in trifluoroethanol, 1.0 mL. To this was added triflic acid, 2.3 μL, and the mixture stirred for 10 minutes. HPLC showed a complete disappearance of the starting material, replaced by a single peak eluting at 10.8 minutes. The mixture was evaporated and redissolved in water and evaporated several times. Yield 2.4 mg 89% based on starting 1,4,7,10-tetraazacyclododecane-1,4,7-triacetic acid, 10-[(2-t-butoxy-5-(N-{CNCbl-5′-[(13-amino)-4,7,10-trioxa-tridecanecarbamate])}-carbonylamino)phenyl)methyl]-, tri-t-butyl ester. Mass spectrometry shows (M+2H)²⁺=1048.4 Theory=1048.6.

### Synthesis of 1,4,7,10-Tetraazacyclododecane-1,4,7-triacetic acid. 10-[(2-hydroxy-5-(N-{N^{ε}-lys(3)-bombesin(1-14)}-carbonylamino)phenyl)methyl](11)

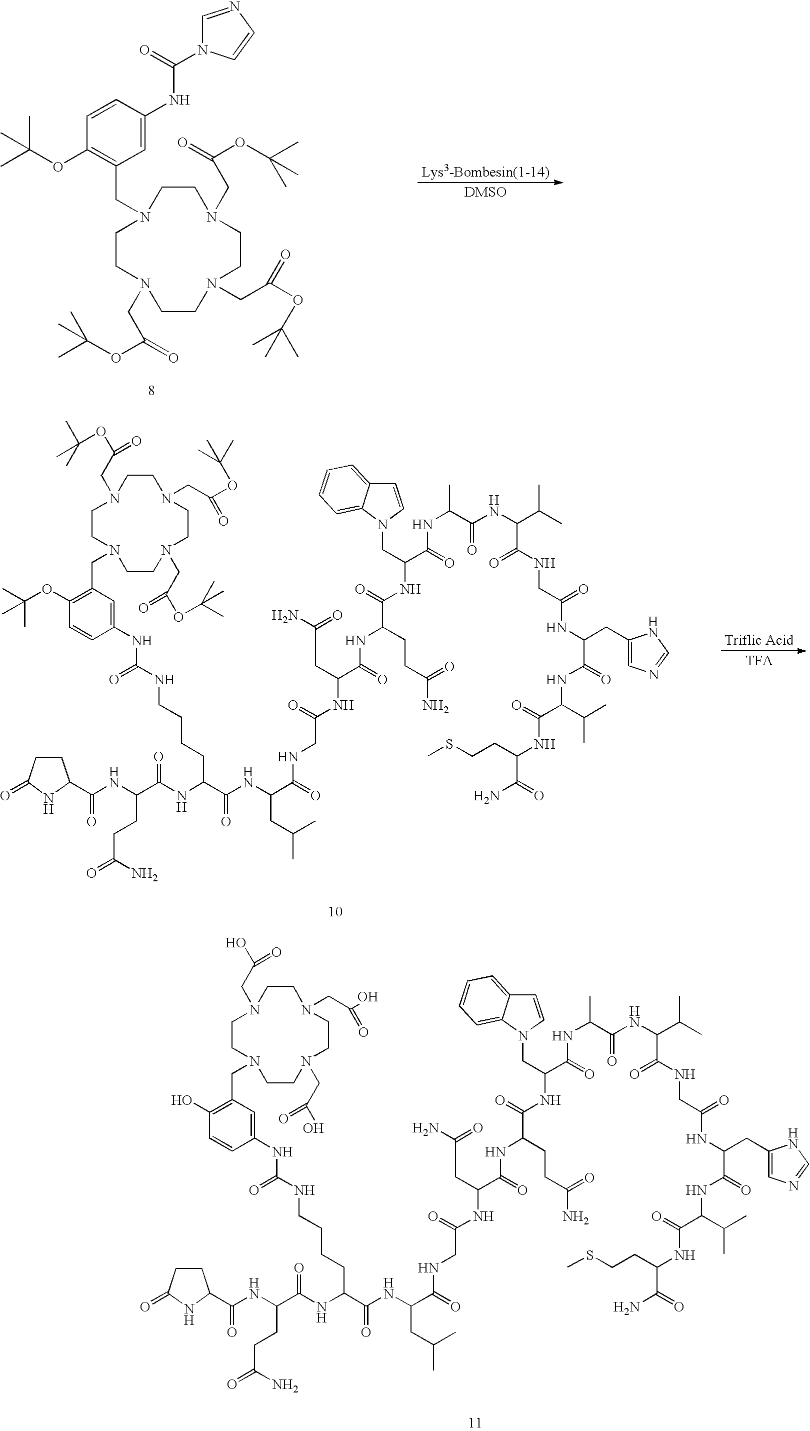

### Synthesis of 1,4,7,10-Tetraazacyclododecane-1,4,7-triacetic acid, 10-[(2-hydroxy-5-(N-{N^{ε}-lys(3)-bombesin(1-14)}-carbonylamino)phenyl)methyl], tri-t-butyl ester (10)

1,4,7,10-Tetraazacyclododecane-1,4,7-triacetic acid, 10-[(2-t-butoxy-5-(imidazolylcarbonylamino)phenyl)methyl]-, tri-t-butyl ester, 3.0 mg 3.8 millimoles, was dissolved in anhydrous DMSO. To this was added lys(3)-bombesin(1-14), 5 mg 3.1 millimoles. The mixture was stirred for four hours. HPLC of an aliquot revealed the reaction was not complete. An additional aliquot of 1,4,7,10-tetraazacyclododecane-1,4,7-triacetic acid, 10-[(2-t-butoxy-5-(imidazolylcarbonylamino)phenyl)methyl]-, tri-t-butyl ester, 1.5 mg 1.9 millimoles, was added and the mixture was allowed to stir overnight. The crude product was isolated by precipitation with diethyl ether and purified by reverse phase chromatography. Yield 3.0 mg 41% based on starting lys(3)-bombesin(1-14). LCMS shows (M+2H)²⁺=1155.6 (Theory=1155.1).

### Synthesis of 1,4,7,10-Tetraazacyclododecane-1,4,7-triacetic acid, 10-[(2-hydroxy-5-(N-{N^{ε}-lys(3)-bombesin(1-14)}-carbonylamino)phenyl)methyl](11)

A sample of 1,4,7,10-tetraazacyclododecane-1,4,7-triacetic acid, 10-[(2-hydroxy-5-(N-{N^{ε}-lys(3)-bombesin(1-14)}-carbonylamino)phenyl)methyl], tri-t-butyl ester, 3.0 mg 0.0013 millimole, was suspended in deionized water, 0.01 mL. To this was added trifluoroacetic acid, 0.5 mL, and the mixture was allowed to stir overnight. The solvent was evaporated and the residue treated with fresh water and evaporated several times. The residue was purified by reverse phase HPLC, 5μ C18. Yield 0.001 g 37% based on starting 1,4,7,10-tetraazacyclododecane-1,4,7-triacetic acid, 10-[(2-hydroxy-5-(N-{N^{ε}-lys(3)-bombesin(1-14)}-carbonylamino)phenyl)methyl], tri-t-butyl ester. LCMS shows (M+2H)²⁺=1043.3 (Theory=1043.0).

1. A conjugate comprising a bio-directing carrier, a metal coordinating moiety, and a linker chemically linking the metal coordinating moiety to the carrier, the linker comprising a urea moiety.

2. The conjugate of claim 1 wherein the bio-directing carrier is selected from the group consisting of imidazole, triazole, antibodies, proteins, peptides, carbohydrates, vitamins, hormones, drugs, and small organic molecules.

3. The conjugate of claim 1 wherein the conjugate comprises more than one bio-directing carrier.

4. The conjugate of claim 1 wherein the metal coordinating moiety is a polycarboxylic acid.

5. The conjugate of claim 3 wherein the metal coordinating moiety is selected from the group consisting of EDTA, DTPA, DCTA, DOTA, TETA, or analogs or homologs thereof.

6. The conjugate of claim 1 wherein the metal coordinating moiety is a triaza- or tetraza-macrocycle.

7. The conjugate of claim 1 wherein the metal coordinating moiety is complexed with a metal, the metal consisting of a radioisotope or a paramagnetic metal.

8. The conjugate of claim 7 wherein the metal is selected from the group consisting of Lu, Lu-177, Y, Y-90, In, In-111, Tc, Tc═O, Tc-99m, Tc-99m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

9. The conjugate of claim 1 wherein the metal coordinating moiety comprises a substituted heterocyclic ring.

10. The conjugate of claim 9 wherein said heterocyclic ring comprises 9 to 15 ring atoms, at least 3 of said ring atoms being nitrogen.

11. The conjugate of claim 9 wherein said heterocyclic ring comprises 3-5 ring nitrogen atoms.

12. The conjugate of claim 9 wherein said heterocyclic ring is optionally substituted at one or more ring carbon atoms.

13. The conjugate of claim 12 wherein said heterocyclic ring is substituted at one or more ring nitrogen atoms.

14. The conjugate of claim 1 wherein the metal coordinating moiety comprises a substituted heterocyclic ring having the following structure: 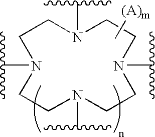
wherein
n is 0, 1 or 2; and
m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio.

15. The conjugate of claim 1 wherein the metal coordinating moiety comprises a substituted heterocyclic ring having the following structure: 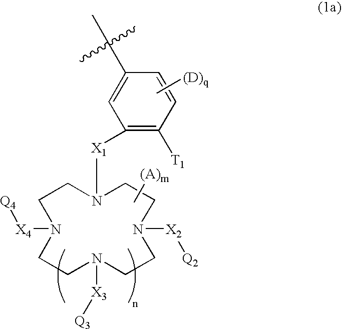
wherein
n is 0, 1 or 2;
m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₄ are independently selected from the group consisting of: 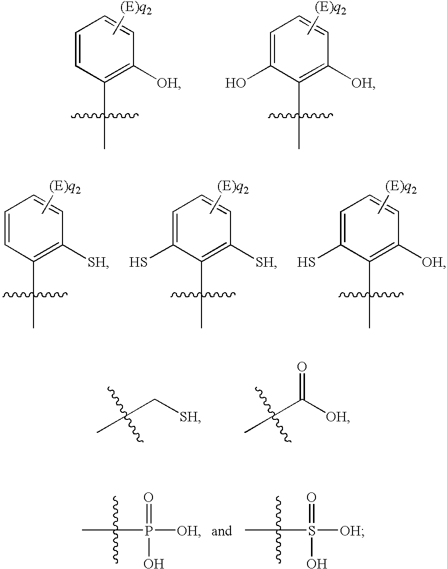
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphate; and
T₁ is hydroxyl or mercapto.

16. The conjugate of claim 1 wherein the metal coordinating moiety comprises a heterosubstituted alkyl chain.

17. The conjugate of claim 16 wherein said heterosubstituted alkyl chain comprises 4 to 10 atoms, at least 2 of said atoms being nitrogen.

18. The conjugate of claim 16 wherein said heterosubstituted alkyl chain comprises 2-4 nitrogen atoms.

19. The conjugate of claim 16 wherein said heterosubstituted alkyl chain is optionally substituted at one or more carbon atoms.

20. The conjugate of claim 19 wherein said heterosubstituted alkyl chain is substituted at one or more nitrogen atoms.

21. The conjugate of claim 1 wherein the metal coordinating moiety comprises a heterosubstituted alkyl chain having the following structure: 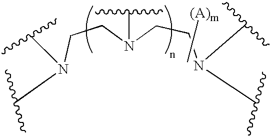
wherein
n is 0, 1 or 2;
m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio.

22. The conjugate of claim 1 wherein the metal coordinating moiety comprises a heterosubstituted alkyl chain having the following structure: 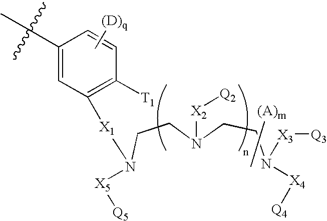
wherein
n is 0, 1 or 2;
m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphate, and phosphito;
X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₅ are independently selected from the group consisting of: 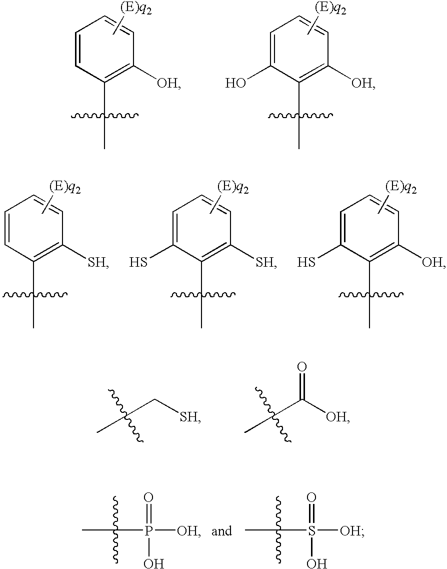
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphate; and
T₁ is hydroxyl or mercapto.

23. The conjugate of claim 15 wherein Q₂-Q₅ are selected from the group consisting of:

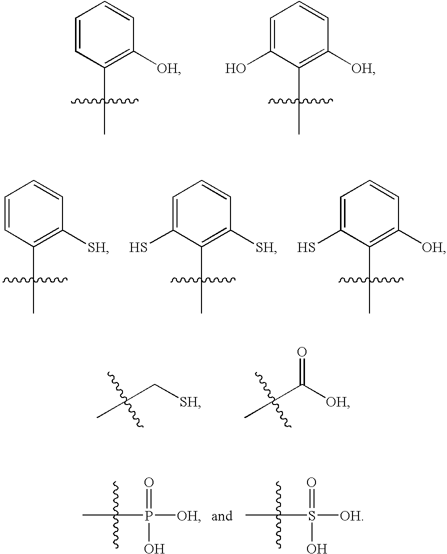

24. The conjugate of claim 1 wherein the linker has the formula: 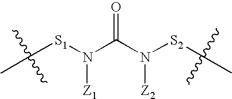
wherein
S₁ and S₂ are spacers, each independently being a bond or a series of atoms, and
Z₁ and Z₂ are independently hydrogen, aryl, C₁₋₇ alkyl, C₁₋₇ hydroxyalkyl or C₁₋₇ alkoxyalkyl.

25. The conjugate of claim 24 wherein S₁ and S₂ are independently a single covalent bond, aryl or C₁₋₂₀ alkylene optionally substituted with one or more carbaldehyde, keto, carboxyl, cyano, halo, nitro, amido, sulfato, sulfito, phosphato, phosphito, hydroxyl, oxy, mercapto, thio, or sulfoxo.

26. The conjugate of claim 1 wherein the metal coordinating moiety is complexed with a metal, M, forming a metal complex having the formula 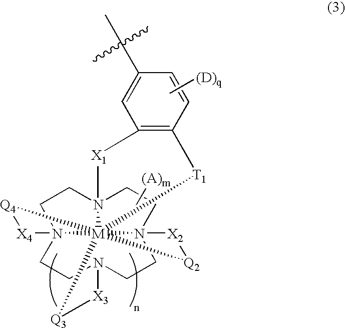
wherein
n is 0, 1 or 2;
m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₄ are independently selected from the group consisting of: 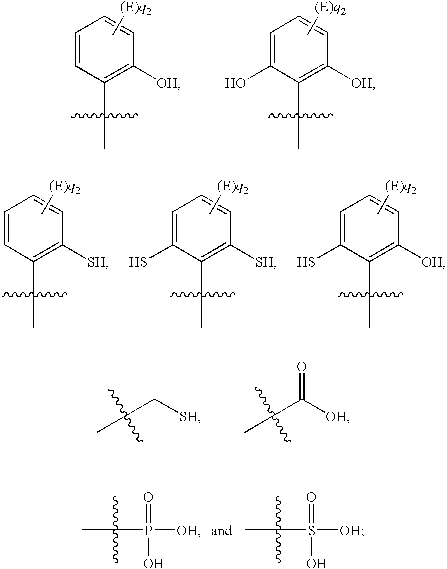
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C1-20 alkyl optionally substituted with one or more or C1-20 alkyl, carboxy, cyano, nitro, amido, hydroxyl, sulfito, phospito, sulfato, and phosphato;
T₁ is hydroxyl or mercapto; and
M is selected from the group consisting of Lu, Lu-177, Y, Y-90, In, In-111, Tc, Tc═O, Tc-99m, Tc-99m═O, Re, Re-186, Re-188, Re═O, Re-186=0, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

27. The conjugate of claim 1 wherein the metal coordinating moiety is complexed with a metal, M, forming a metal complex having the formula 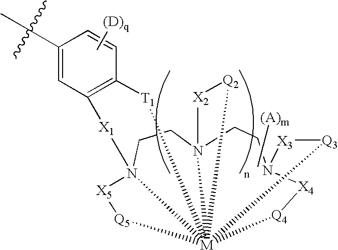
wherein
n is 0, 1 or 2;
m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄ and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₅ are independently selected from the group consisting of: 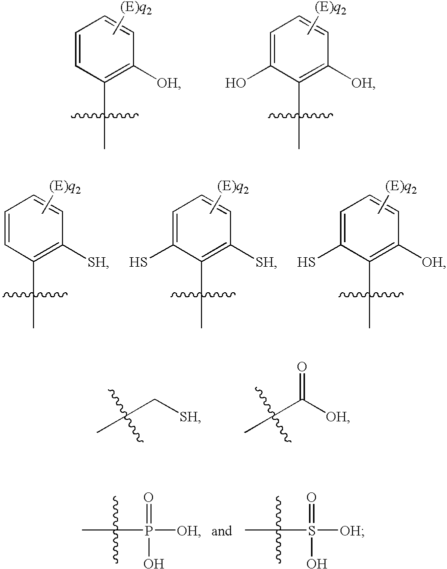
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C1-20 alkyl optionally substituted with one or more or C1-20 alkyl, carboxy, cyano, nitro, amido, hydroxyl, sulfito, phospito, sulfato, and phosphato;
T₁ is hydroxyl or mercapto; and
M is selected from the group consisting of Lu, Lu-177, Y, Y-90, In, In-111, Tc, Tc═O, Tc-99m, Tc-99m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

28. A pharmaceutical composition comprising the conjugate of claim 1 and a pharmaceutically acceptable carrier.

29. A method for the diagnosis of cancer in a mammal, the method comprising administering to said mammal an effective amount of the conjugate of claim 1 for the diagnosis of cancer and a pharmaceutically acceptable carrier.

30. A method for treating cancer in a mammal, the method comprising administering to said mammal an effective amount of the conjugate of claim 1 and a pharmaceutically acceptable carrier.

31. A kit comprising a protected metal coordinating moiety, an active urea, a deprotecting acid, a buffer, and a solution of a radioactive metal.

32. The kit of claim 31 wherein the metal coordinating moiety comprises a substituted heterocyclic ring having the following structure: 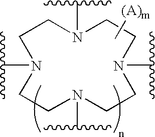
wherein
n is 0, 1 or 2; and
m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphate, phosphito, hydroxyl, oxy, mercapto or thio.

33. The kit of claim 31 wherein the metal coordinating moiety comprises a substituted heterocyclic ring having the following structure: 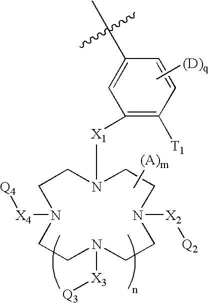
wherein
n is 0, 1 or 2;
m is 0-16 wherein when m is greater than 0, each A is C₁₋₂₀ alkyl or aryl optionally substituted by one or more aryl C₁₋₂₀ alkyl, carbaldehyde, keto, carboxyl cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₄ are independently selected from the group consisting of: 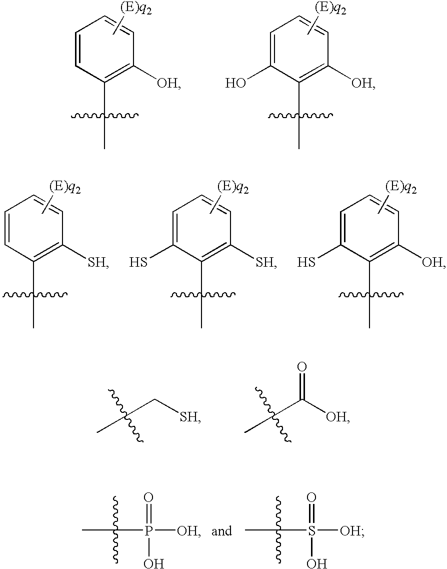
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato; and
T₁ is hydroxyl or mercapto.

34. The kit of claim 31 wherein the metal coordinating moiety comprises a heterosubstituted alkyl chain having the following structure: 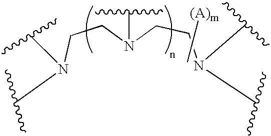
wherein
n is 0, 1 or 2;
m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio.

35. The kit of claim 31 wherein the metal coordinating moiety comprises a heterosubstituted alkyl chain having the following structure: 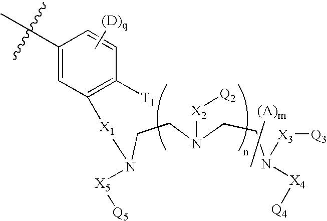
wherein
n is 0, 1 or 2;
m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphate, phosphito, hydroxyl, oxy, mercapto and thio;
Q₂-Q₅ are independently selected from the group consisting of: 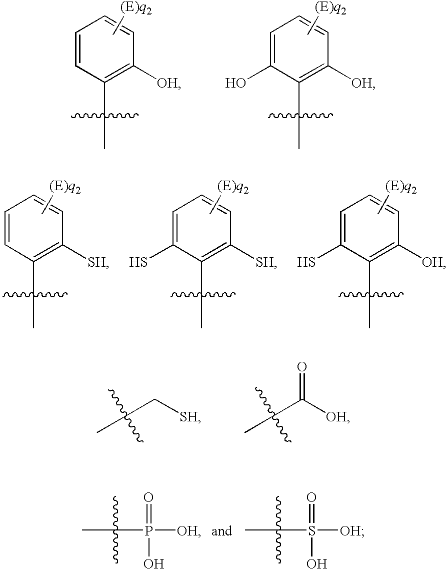
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C1-20 alkyl optionally substituted with one or more or C1-20 alkyl, carboxy, cyano, nitro, amido, hydroxyl, sulfito, phospito, sulfato, and phosphate; and
T₁ is hydroxyl or mercapto.

36. The kit of claim 31 wherein the radioactive metal is selected from the group consisting of Lu, Lu-177, Y, Y-90, In, In-111, Tc, Tc═O Tc-99m, Tc-99m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

37. The kit of claim 31 wherein the buffer is selected from the group consisting of citrate, phosphate and borate.

38. The kit of claim 31 wherein the metal coordinating moiety, the active urea, the deprotecting acid, the buffer, and the solution of a radioactive metal are in unit dosage form.
